(19) United States
(12) Patent Application Publication
(10) Pub. No.: US 2016/0228066 A1
(43) Pub. Date: Aug. 11, 2016

(54) BINARIZED FREQUENCY TRANSFORM

(71) Applicant: INTEL CORPORATION, Santa Clara, CA (US)

(72) Inventors: Steven P. Szabados, San Francisco, CA (US); Andrew A. Stirn, San Francisco, CA (US)

(73) Assignee: INTEL CORPORATION, Santa Clara, CA (US)

(21) Appl. No.: 15/022,051

(22) PCT Filed: Nov. 19, 2014
(86) PCT No.: PCT/US14/66454

§ 371 Date: Mar. 15, 2016

Related U.S. Application Data
(60) Provisional application No. 61/906,829, filed on Nov. 20, 2013.

(51) Int. Cl. A61B 5/00; G06F 17/30; G06F 19/00; A61B 5/0205

(52) U.S. Cl. CPC A61B 5/7253; A61B 5/02055; A61B 5/681; G06F 17/30569; G06F 19/3406; A61B 5/0531

(57) ABSTRACT

A computing device analyzes data taken over a period of time using a data transform. Frequency components are extracted by converting the data to a binary signal and computing an approximate inner product between the converted data and one or more basis functions. To compute the inner product between the converted data and a basis function, an exclusive nor (XNOR) function is used to perform element-wise multiplication between the converted data and the basis function, and a lookup table is used to sum the resulting products. Each resulting sum corresponds to an approximation of the basis function's strength in the data. When binary sine and cosine functions are used, the method approximates the discrete Fourier transform. Other basis functions may be used to approximate various transforms.

### CROSS-REFERENCE TO RELATED APPLICATION

This application claims the benefit of U.S. Provisional Application No. 61/906,829, filed Nov. 20, 2013, which is incorporated by reference in its entirety.

### BACKGROUND

1. Field of Art

The disclosure generally relates to the field of physiological monitoring, and more particularly to the field of processing physiological data.

2. Description of the Related Art

Physiological monitoring devices monitor physiological parameters such as human heart rate, motion, skin conductivity, and body temperature. Since these devices are initially developed for use in a clinical setting, they lack portability because they are heavy, bulky, and restrict human movement. To enable physiological monitoring outside of a clinical setting, portable physiological sensors have been developed. However, many portable monitoring devices do not provide timely feedback to the user about multiple physiological parameters or have limited battery power.

Some physiological monitoring devices analyze the frequency of a physiological signal to determine more meaningful physiological parameters. For example, the Fourier transform of a user's cardiac data transforms the physiological signal into the frequency domain, which has a dominant peak at a frequency corresponding to the user's heart rate. However, determining the Fourier transform of any signal is a computationally intensive process due to the use of matrix multiplication, which is computationally expensive to perform in a processor. To improve computational efficiency, many processors include a dedicated circuit to perform matrix multiplications (or even a coprocessor such as a Graphics Processing Unit). However, a dedicated multiplier circuit (or coprocessor) increases power consumption and accordingly reduces battery life. To compensate for reduced battery life, battery size and weight may be increased, but bulky batteries decrease the portability of the physiological monitoring device. Accordingly, present physiological monitoring devices face a disadvantageous tradeoff between mobility and battery endurance.

### BRIEF DESCRIPTION OF DRAWINGS

The disclosed embodiments have other advantages and features which will be more readily apparent from the detailed description, the appended claims, and the accompanying figures (or drawings). A brief introduction of the figures is below.

FIG. 1A illustrates one embodiment of an example wearable device that is configured to be in close proximity to or in contact with a user.

FIG. 1B illustrates one embodiment of an example wearable device from a side perspective.

FIG. 2 illustrates one embodiment of a sensor panel of the wearable device.

FIG. 3 illustrates a block diagram of an example system for processing sensor data in one embodiment.

FIG. 4 illustrates a flow chart of an example process for extracting frequency information from time-based data in one embodiment.

FIG. 5 illustrates a process flow diagram of an example process for converting recorded data to a binary signal in one embodiment.

FIG. 6 illustrates a process flow diagram of an example process for multiplying binary data in one embodiment.

FIG. 7 illustrates a process flow diagram of an example process for tallying binary data in one embodiment.

FIG. 8A illustrates an example process for converting example data to a binary signal by comparing a first-derivative of the example data to a threshold in one embodiment.

FIG. 8B illustrates an example process for converting an example function to a binary signal by comparing the example function to a threshold in one embodiment.

FIG. 9 illustrates an example basis functions from a basis function store in one embodiment.

FIG. 10 illustrates example entries of a bit sum store in one embodiment.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Configuration Overview

One embodiment of a system, method, and a computer-program product (e.g., a computer-readable storage medium storing instructions or computer program code) is disclosed that includes a wearable device having one or more sensors and instructions that cause a processor on the wearable device to extract frequency information from data gathered by the one or more sensors. The wearable device is attached to a user with a fastening system and measures physiological data using sensors. Gathered physiological data is shown on a display or screen. The wearable device may also include interaction points for the user to select data for display, to organize data, or to modify preferences regarding data. Included sensors measure user motion, blood flow characteristics, temperature, and skin conductivity. Collected physiological data is processed and/or is stored using a microcontroller.

The microcontroller may process data to extract frequency information for determining heart rate, beat-to-beat variance, respiration, beat-to-beat magnitude, and beat-to-beat coherence. As another example, the microcontroller can use frequency extraction to count steps, analyze gait, or determine pacing information about a user of the wearable device. Example applications of the disclosed binary data transform also include audio signal processing (e.g., detecting touch tones in a telephone signal), spectral estimation (e.g., spectroscopy), image processing (e.g., image compression or reconstruction), video processing (e.g., compression), financial signal processing (e.g., detecting regular patterns), and data analysis (e.g., digital signal processing, noise elimination, characterizing turbulence in a fluid, detecting periodic clustering in a field of particles, earthquake detection).

To extract frequency components, data is converted to a binary signal, windowed, and an approximate inner product of the data with a set of basis functions is computed. More broadly, data may be approximated as a weighted sum of a set of basis functions. A set of basis functions contains variations on one or more functions. One example set of basis functions contains binary sine waves and cosine waves with frequencies that are an integer multiple of a base frequency. Other example basis functions include pulse trains, triangular waves, sawtooth waves, wavelets, chirps, chirplets, and cosine-and-sine functions. For general basis functions, the inner product of data against a basis function indicates the relative weights of each basis function in a linear combination of those basis functions to approximate the data.

When a sine or cosine wave having a particular frequency is used as a basis function, the inner product indicates the strength of the particular frequency in the collected data. When the inner product is calculated using binary sines and cosines at discrete frequencies across a spectrum, the process performs an approximation of a discrete Fourier transform. Taking the inner product comprises multiplication and summation operations. The multiplication operation may be performed using an exclusive nor logical operation (XNOR) between the binary data and a binary representation of the basis function. The summation operation may be performed with a lookup table to reduce addition operations. The extracted frequency data may be further processed (e.g., to determine heart rate), displayed, and/or stored. The disclosed embodiments improve on prior embodiments by reducing the hardware needed to perform multiplication by performing binary multiplication using an XNOR. Also, the use of a particular number of samples in the data window fixes the values of the basis function used in the multiplications. Because the values of the basis function are fixed, they may be stored in a lookup table or other hardware embodiment, which avoids repeating hardware-intensive multiplication, division, and trigonometric operations.

### Wearable Physiological Measuring Device

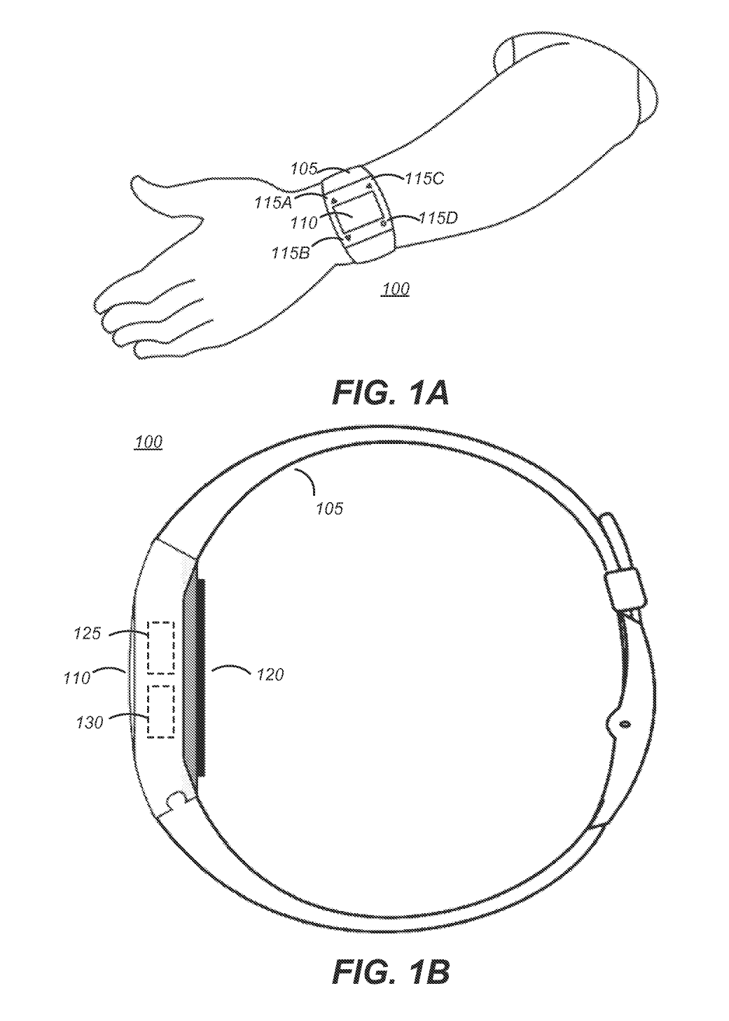

The disclosed method for a binarized data transform is exemplified in reference to a device that monitors physiological data. FIG. 1A illustrates an example of a wearable device 100 configured to be in close proximity to or in contact with a user. For example, the device 100 may be worn on a user's appendage (e.g., an arm, a wrist, an ankle) or portion thereof. In one embodiment, the wearable device 100 is a physiological monitoring device for monitoring activities of its wearer and calculating various physiological and kinematic parameters, such as activity levels, caloric expenditure, step counts, heart-rate, body temperature, ambient temperature, perspiration, and sleep patterns. A fastening system 105 fastens the device 100 to a user's appendage. The fastening elements 105 may be removable, exchangeable, or customizable. One example fastening element includes a band around the user's appendage held in place through a buckle, a clasp, a hook, a pin, a hook-and-loop fastener, an elastic compression fastener, or magnets. Although embodiments are described herein with respect to a wrist-worn device, other form factors or designed wear locations of the wearable device 100 may alternatively be used. For example, embodiments of the method described herein may be implemented in arm-worn devices, head-worn devices, clip-on devices, and so forth.

The wearable device 100 includes a display 110 and several user interaction points 115A, 115B, 115C, 115D. The display 110 and user interaction points 115 may be separate components of the device 100, or may be a single component. For example, the display 110 may be a touch-sensitive display configured to receive user touch inputs and display information to the user. The wearable device may also have a display such as 110 without interaction points, or interaction points 115 without a display element such as 110. Embodiments of the display 110 include a liquid crystal display (LCD), a light-emitting diode (LED) display, and an organic LED display. As an alternative to the display 110 (or in addition), the wearable device includes a screen. In an embodiment, the display 110 shows various pieces of information to a user such as the biometric data measured by sensors. Embodiments of the interaction points 115 include buttons, dials, and capacitive sensors.

FIG. 1B is a side perspective of an embodiment of the device 100, showing the fastening system 105, the display 110, a sensor panel 120, one or more processors 125, and a memory 130. The sensor panel 120 contains one or more sensors for monitoring user physiology, kinematic parameters, or ambient conditions. Example measurements include blood flow, device motion, perspiration, or temperature, in an embodiment. The sensor panel 120 is communicatively coupled to the one or more processors 125 and/or memory 130. The one or more processors 125 execute instructions, prepare data for display, and process sensor data. Embodiments of the processor 125 include a microprocessor, a central processing unit (CPU), a graphics processing unit (GPU), a digital signal processor (DSP), one or more application specific integrated circuits (ASICs), one or more field programmable gate arrays (FPGAs), one or more programmable logic devices (PLDs), or any combination of these. The memory 130 stores data collected from the sensor panel 120 and stores instructions for execution by the one or more processors 125. Embodiments of the memory 130 include flash memory, random-access memory (RAM), read-only memory (ROM), or a combination thereof. The memory 130 includes a machine-readable medium on which instructions (e.g., software) embodying any one or more of the methodologies or functions described herein are stored. The instructions may also reside, completely or at least partially, within the one or more processors 125 (e.g., within a processor's cache memory) during execution thereof. In an embodiment, the functionality of the one or more processors 125, memory 130, and other components is provided by a component such as a microcontroller, a system on a chip (SoC), a programmable SoC (PSoC), a programmable logic relay (PLR), or a programmable logic controller (PLC).

It should be noted that the wearable device 100 may include additional components not shown in FIGS. 1A or 1B. In an embodiment, the device includes components such as vibratory motors, electroactive polymers, or piezoelectric surfaces for haptic feedback. The wearable device may include network interface devices such as a universal serial bus (USB) port or wireless transceivers for communication using protocols such as WiFi, Bluetooth, Bluetooth Low Energy (BTLE), Adaptive Network Topology (ANT), and Zigbee. In an embodiment, the wearable device 100 may be connected (e.g., networked) to other machines. In a networked deployment, the wearable device may operate in the capacity of a client machine in a server-client network environment, or as a peer machine in a peer-to-peer (or distributed) network environment.

In an embodiment, the device includes a power supply such as a battery, a motion-powered generator, or a solar generator. Embodiments may include an audio signaling device or additional visual indicators such as LEDs for providing feedback to a user. Embodiments may include electronic filters, amplifiers, power source regulators, analog-to-digital converters (ADC), digital-to-analog converters (DAC), and other circuitry.

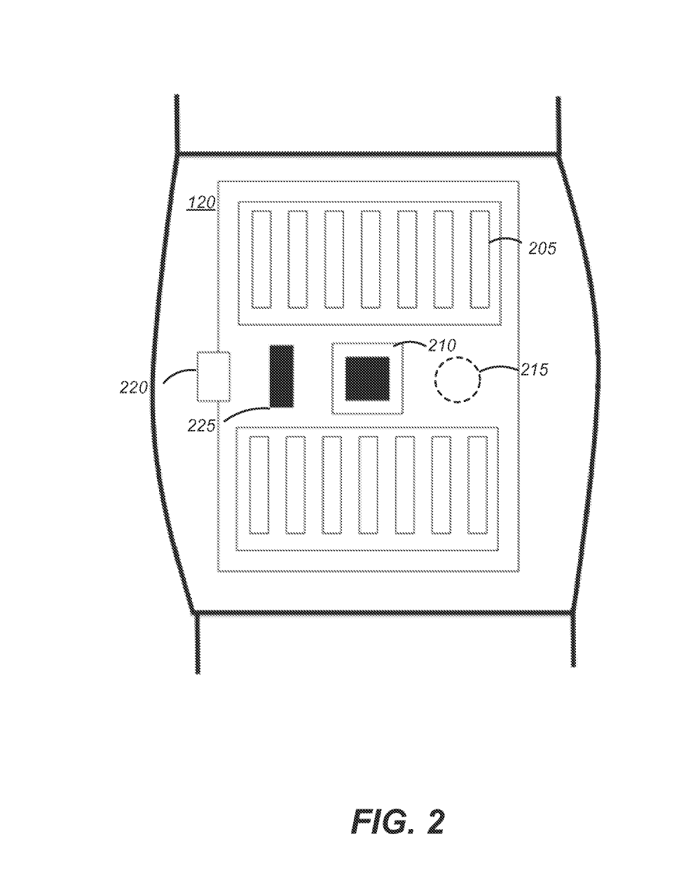

Referring to FIG. 2, illustrated is an embodiment of the sensor panel 120, shown from a side (e.g., an underside) of the wearable device 100 opposite the display 110. The sensor panel 120 includes a skin conductivity sensor 205, an optical sensor 210, a multi-axis motion sensor 215, an ambient thermal sensor 220, and a skin thermal sensor 225. Embodiments may include a combination of some or all of the previously listed sensors and/or additional sensors (e.g., a hygrometer for measuring humidity, a barometer for measuring pressure, a photodetector for measuring ambient light). Sensors may be configured or mounted elsewhere on the wearable device, Alternatively or additionally.

The skin conductivity sensor 205 measures conductivity of skin using an ohmmeter, in an embodiment. A differential voltage is applied between two terminals, and current is measured. Conductance may be inferred based on distance between terminals, applied voltage, and measured current. Conductance readings may be based on multiple measurements between additional terminals. Skin conductance measurements may be adjusted based on readings from the ambient thermal sensor 220, the skin thermal sensor 225, or other factors. Skin conductivity measurements can detect perspiration or emotional arousal.

The optical sensor 210 measures heart rate of a user by measuring a rate of blood flow, in one embodiment. An emitter of the optical sensor 210 sends a light signal to skin and tissue of the wearer of the device 100 and measures the amount of light reflected to a detector. A portion of the light signal emitted by the emitter is absorbed by the wearer's tissue and a portion is reflected to the detector. In particular, if the light is of a wavelength absorbed by blood, a portion of the light is absorbed by the wearer's blood. Thus, the amount of light reflected to the detector depends in part on the volume of blood under the skin. As blood volume cyclically changes due to cardiac cycles, the amount of light detected by the detector cyclically varies. The detector converts the measured light intensity into a voltage, which is analyzed for regular variations that indicate the rate of the heart's pumping cycles. Example biometric data inferred from the optical sensor 210 include pulse rate, beat-to-beat variance, respiration, beat-to-beat magnitude, and beat-to-beat coherence.

The multi-axis motion sensor 215 senses kinematic information about the wearable device 100 such as position, velocity, acceleration, orientation, angular velocity, and angular acceleration. Example multi-axis motion sensors 215 include accelerometers, gyroscopes, and magnetometers. The multi-axis motion sensor 215 can comprise one or more of these. Additionally or alternatively, a global positioning system (GPS) may be used. The multi-axis motion sensor 215 can infer step counts, activity levels, or user speed, for example.

The ambient thermal sensor 220 and skin thermal sensor 225 measure temperature of the ambient environment and the user's skin, respectively. In an embodiment, the thermal sensors 220 and 225 include one or more thermocouples and/or thermistors. By monitoring the resistance (or conductance) of the thermistor, the temperature of the thermistor may be deduced from the thermistor's material properties.

### Architecture for Processing Sensor Data

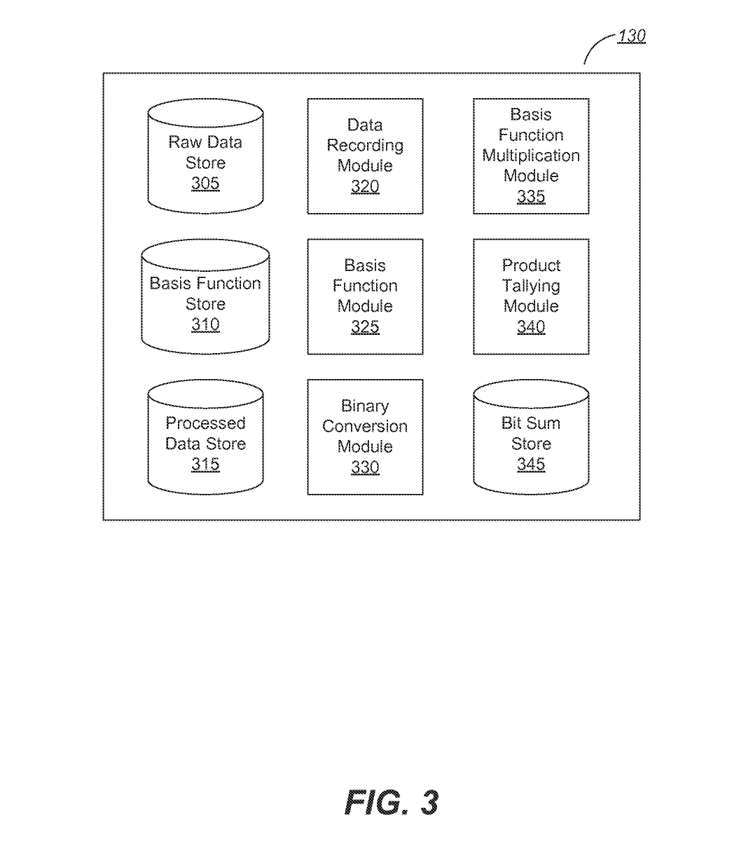

Referring to FIG. 3, illustrated is a block diagram of an example system for processing sensor data. In one embodiment, the system includes modules represented by instructions or computer code stored on the memory 130 and executable by the processor 125. Alternatively or additionally, the functionality of the modules is embodied in a hardware circuit. The modules include a raw data store 305, a basis function store 310, a processed data store 315, a data recording module 320, a basis function module 325, a binary conversion module 330, a basis function multiplication module 335, a product tallying module 340, and a bit sum store 345. Some embodiments may have additional of fewer components. In some embodiments, one or more components may be replaced by fewer or more components together having the same function as the replaced components. The disclosed stores and modules may be implemented using instructions stored in the memory 130 and executed by the one or more processors 125.

The raw data store 305 holds data collected by sensors of the wearable device 100, in an embodiment. The raw data may comprise digital samples of analog signals converted using a digital-to-analog converter. In an embodiment, the data recording module 320 records data from sensors and stores the data in the raw data store 305. In an embodiment, data is processed directly for storage or display to a user of the wearable device without storage as raw data. The processed data store 315 stores processed data after processing for conversion from voltage to a reading such as blood flow rate, perspiration levels, body temperature, ambient temperature, heart rate, or other physiological measurements.

The basis function used in the transform is stored in the basis function store 310. Basis functions may be used to approximate a signal as a sum of one or more basis functions. For example, the discrete Fourier transform approximates a set of data as a sum of a discrete number of sine and cosine functions of increasing frequency. A number of samples are collected during a time period and analyzed to determine prevalent frequencies over that time period. Alternatively or additionally, different basis functions may be used. For example, the discrete cosine transform uses a basis function based on a cosine. As another example, the discrete Hartley transform uses a sum of a cosine and a sine as a basis function to determine frequencies of a signal. As a third example, the wavelet transform uses wavelets as a basis function to extract time-dependent frequency information with better time resolution at high frequencies than are attainable with the discrete Fourier transform. In an embodiment, the one or more basis functions are converted to a binary representation and stored in the basis function store 310, as described below.

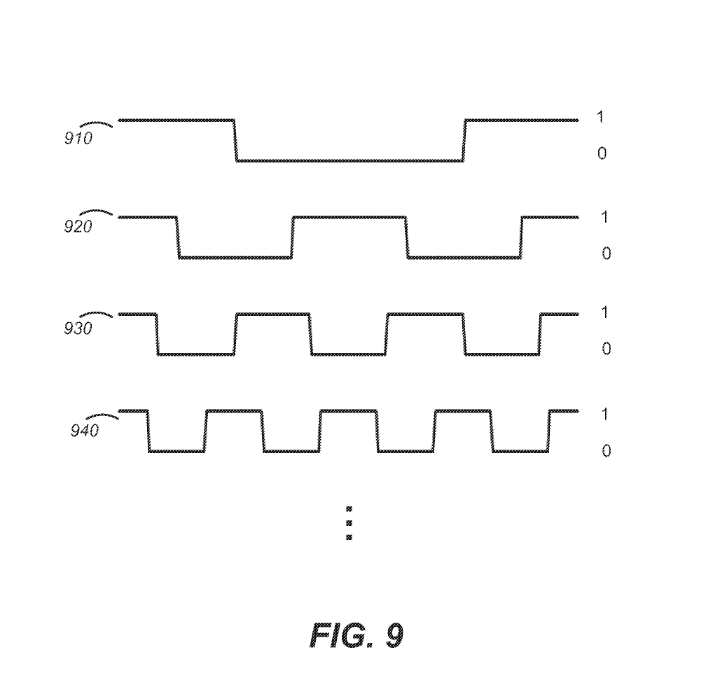

In one embodiment, the basis function store 310 is implemented as an array of basis functions of increasing frequency. One dimension of the array represents a time dimension, and the second dimension of the array represents a frequency dimension. For example, in an approximation of the Fourier transform, one array is used for the binary sine basis function and another is used for the binary cosine basis function. The first row of the binary sine array gives the values of one period of a binary sine wave. The second row contains the values of two periods of a binary sine wave having twice the frequency of the first binary sine wave, and so forth. A column of the binary sine array gives the values at a particular point in time of binary sine waves of linearly increasing frequency down the column. The binary cosine array is similar to the binary sine array except the values of binary cosine waves are stored. A graphical representation of a binary cosine array is described below in conjunction with FIG. 9. Alternatively or additionally, one array having complex number values is used in place of two arrays having real values. The basis function module 325 generates basis functions to be stored in the basis function store 310 for performing the desired transformation. In an embodiment, the generated basis functions are converted to a binary representation, as described below, and then stored. In an embodiment, the basis function module 325 is not included, and the basis functions are stored in the basis function store 310, in the memory 130, or using other hardware and/or firmware of the wearable device 100.

The data recording module 320 records data from the sensor panel 120 and stores the data in the raw data store 305, in one embodiment. In the example device, the sensor panel 120 collects physiological data from a time-based signal. The data recording module 320 handles sampling of the time-based signal at a regular interval to create a digital stream of samples. The data recording module 320 may control an analog-to-digital converter (ADC) in one embodiment. For example, the data recording module 320 samples data from the optical sensor 210. The gathered data is analyzed to infer the heart rate or respiration rate of the wearable device's user. In one embodiment, the data from the data recording module 320 is analyzed substantively in real time in addition to (or as an alternative to) storing the data in the raw data store 305.

The binary conversion module 330 converts recorded data to a binary signal. For example, the binary conversion module 330 may convert digital samples from voltage readings to either a zero or a one. Data samples may be converted based on a logical determination. For example, input values greater than a threshold are set to one and otherwise set to zero. As another example, if the discrete derivative of an input value is greater than a threshold, then the output is set to one; otherwise, the output is set to zero. Alternatively or additionally, arbitrary high and low values are used in place of one and zero. Alternatively or additionally, arbitrary-order derivatives or anti-derivatives of data are compared to a threshold as a basis for binary conversion. In an embodiment, the binary conversion module 330 may further apply a window to received data. A window function selects an integer number N of consecutive samples from an input stream of data, in one embodiment.

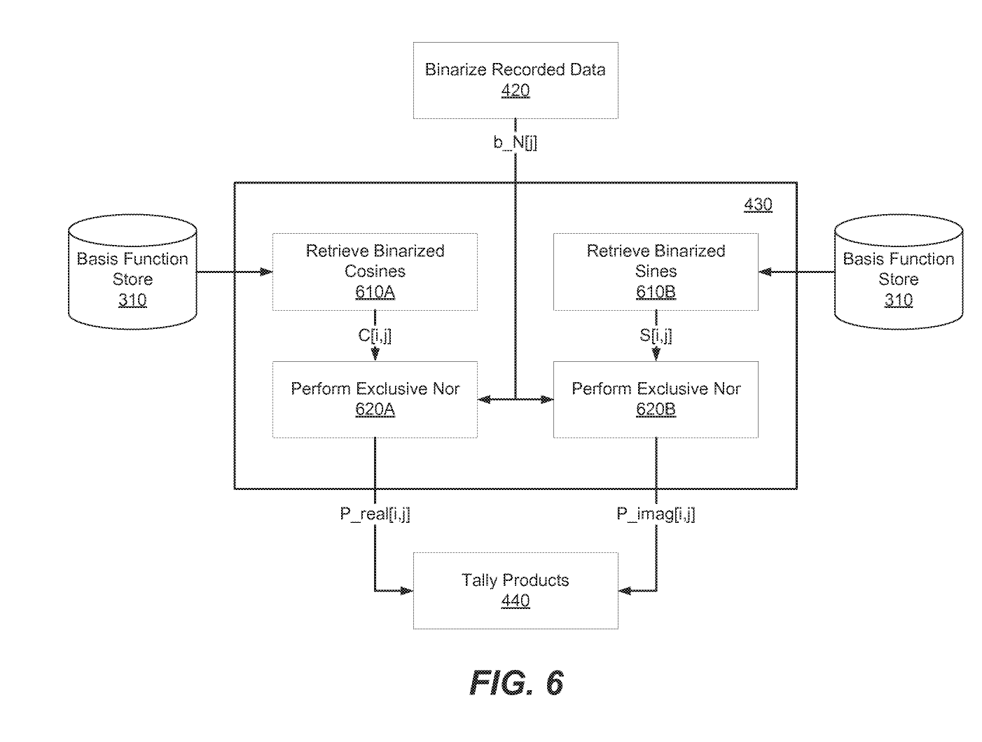

The basis function multiplication module 335 multiplies data element-wise against a basis function. For an approximation of the discrete Fourier transform, input data is multiplied by binary sine and binary cosine basis functions. The binary cosine basis functions are used to determine the real component of frequency amplitude, and the binary sine basis functions are used to determine the imaginary component of frequency amplitude. The results of the binary cosine and binary sine multiplications are two matrices, one matrix for each set of basis functions. In one embodiment, the basis function multiplication module 335 takes as input binarized data from the binary conversion module 330 and uses an exclusive nor (XNOR) to multiply the binarized data with one or more binarized basis functions, as described further in conjunction with FIG. 6.

The product tallying module 340 computes approximate frequencies using the products determined by the multiplication module 335. Tallying products refers to taking a bit sum of products corresponding to a basis function. The bit sum operation is a conditional sum that consolidates the products for a bit sum into a result that indicates the basis function's weight in a linear combination of basis functions to approximate the data. For example, when performing an approximation of the discrete Fourier transform, the bit sum result indicates the approximate strength of a frequency component in the originally recorded data. In the example, the product tallying module 340 determines a bit sum result for each binary sine or cosine used as a basis function. In one representation of the approximate discrete Fourier transform, the product tallying module 340 consolidates two product matrices corresponding to products with binary sine and cosine basis functions into two vectors. The two vectors indicate the imaginary and real components, respectively, of the approximate amplitudes at each basis function frequency.

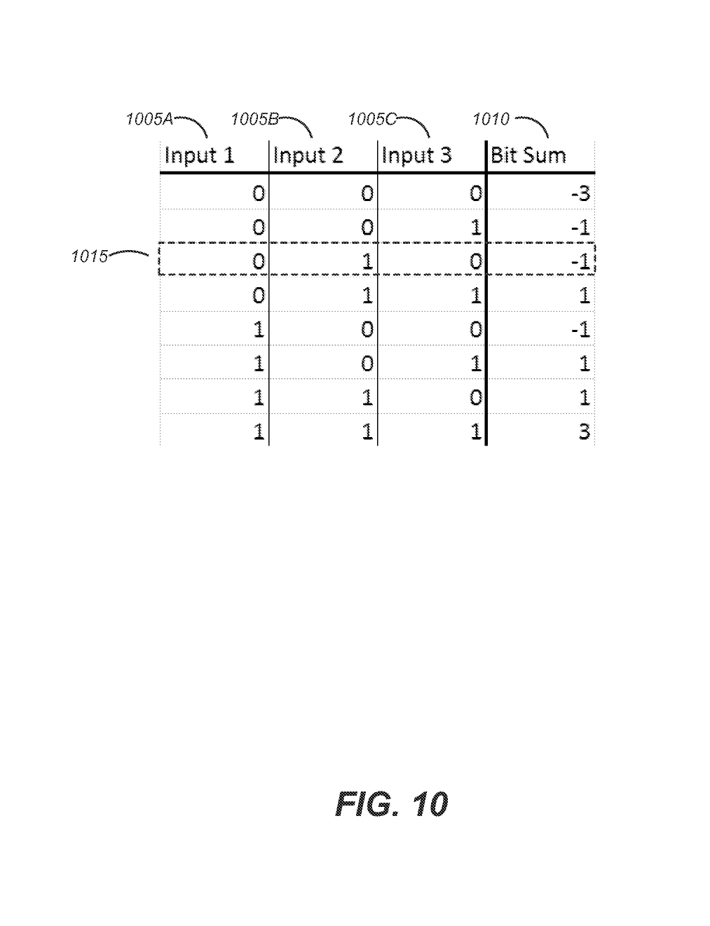

The product tallying module 340 may use the bit sum store 345 to perform bit sum operations. The bit sum store 345 reduces the operations necessary to tally the products. In one embodiment, the bit sum store 345 is a lookup table that matches an input vector of products with a calculated bit sum output. The calculated bit sum output depends on the weights chosen for the bit sum function. FIG. 10 illustrates an example bit sum store 345 and is described in further detail below.

### Example Method

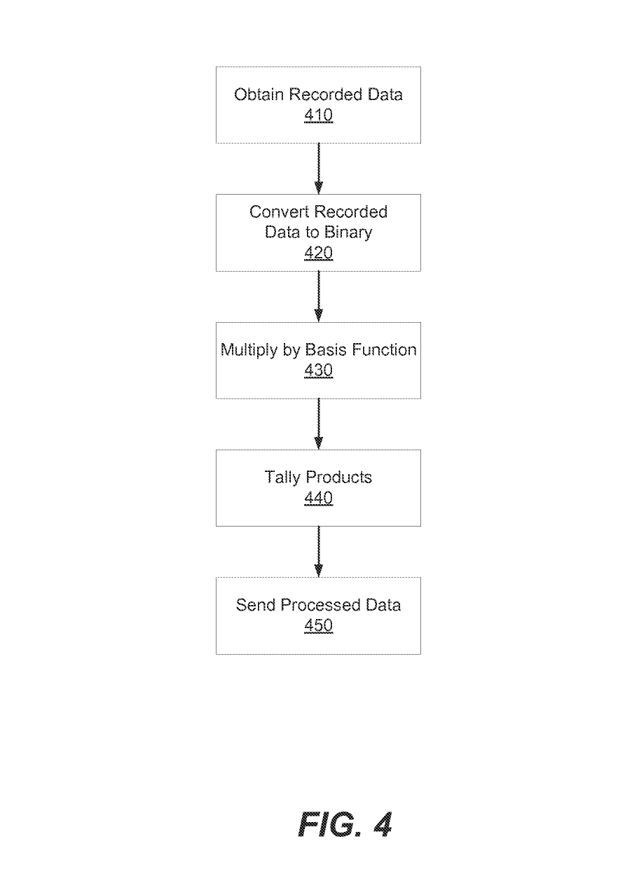

Referring to FIG. 4, illustrated is a flow chart of an example process for extracting frequency information from time-based data. In other embodiments, the data transform process 400 may include different and/or additional steps than those shown in FIG. 4. The data recording module 320 obtains 410 recorded data. In one embodiment, recorded data is obtained 410 from the raw data store 305. Alternatively or additionally, the data recording module 320 obtains 410 recorded data substantially in real time from one or more sensors of the sensor panel 120. In one embodiment, the data recording module 320 samples an analog signal at a regular sampling frequency using an ADC.

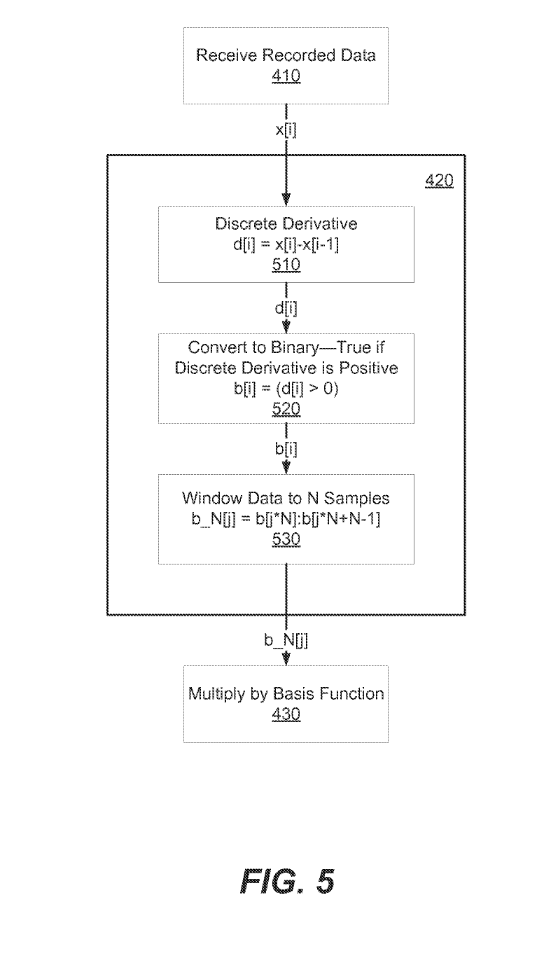

The binary conversion module 330 converts 420 the recorded data to a binary signal. Converting the recorded data to a binary signal includes converting samples of data having integer, decimal, or floating point values into binary values according to a binary conversion process. In an embodiment, converting recorded data to a binary signal may include windowing data into a limited (e.g., windowed) vector of N samples. Converting the recorded data to a binary signal is described further in conjunction with FIG. 5.

The basis function multiplication module 335 multiplies 430 the converted binary data by one or more basis functions. For example, for an approximation of the discrete Fourier transform, the basis function multiplication module 335 multiplies 430 a vector of N samples by N binary cosine basis functions and N binary sine basis functions. Multiplying the converted binary data by one or more basis functions is described further in conjunction with FIG. 6.

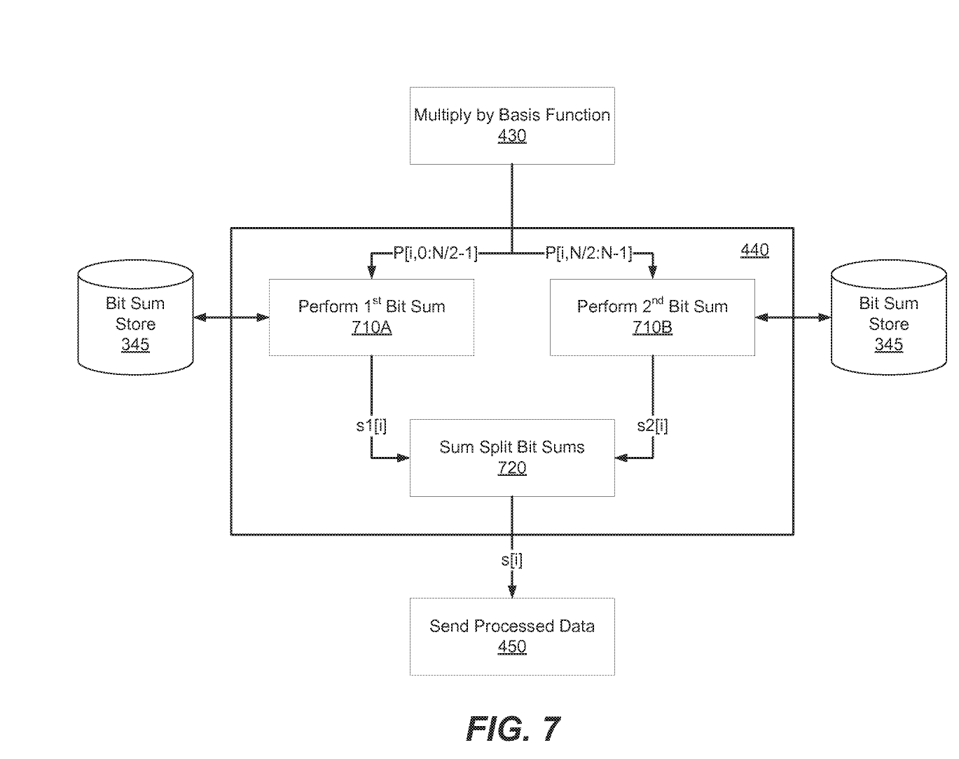

The product tallying module 340 tallies 440 the products of the multiplication to achieve a result indicating the basis functions' relative weighting in a linear combination of basis functions to approximate the recorded data. For example, for an approximation of the discrete Fourier transform, the resulting sums are contained in a first vector of N values representing the real component of frequency amplitudes and a second vector of N values representing the imaginary component of frequency amplitudes. Tallying the products of multiplication is described further in conjunction with FIG. 7.

Together, multiplying 430 data element-wise by a basis function and then tallying 440 the resulting products approximates an inner-product (e.g., a dot product) between the basis function and the binary data, thereby transforming the recorded data from the temporal domain to a domain of the basis function. For example, if the basis function is a cosine, the recorded data is transformed into the frequency domain. The product tallying module 340 sends 450 the processed data to the processed data store 315, in one embodiment. Alternatively or additionally, the product tallying module 340 sends 450 the processed data over a network or to another module for use by another process (e.g., for viewing on the display 110).

Referring to FIGS. 5-7, illustrated are example process flow diagrams for performing the data transform process 400. FIGS. 5-7 illustrate an example implementation of the data transform process 400 for an approximation of the discrete Fourier transform. Alternative embodiments of the data transform process 400 may be based on a different binary conversion criterion or different basis functions.

FIG. 5 illustrates an implementation of converting 420 recorded data to a binary signal. The binary conversion module 330 receives 410 a recorded data signal x[i] and takes 510 the discrete derivative d[i]. The discrete derivative d[i] may be found from the difference of a sample of the recorded data signal x[i] and the previous sample of the recorded data signal x[i-1]. A smoothing function (e.g., a moving average) may be applied before taking 510 a discrete derivative. Received data (or sections thereof) may be fit to a function (e.g., a cubic spline) and derivatives may be determined from the data fit.

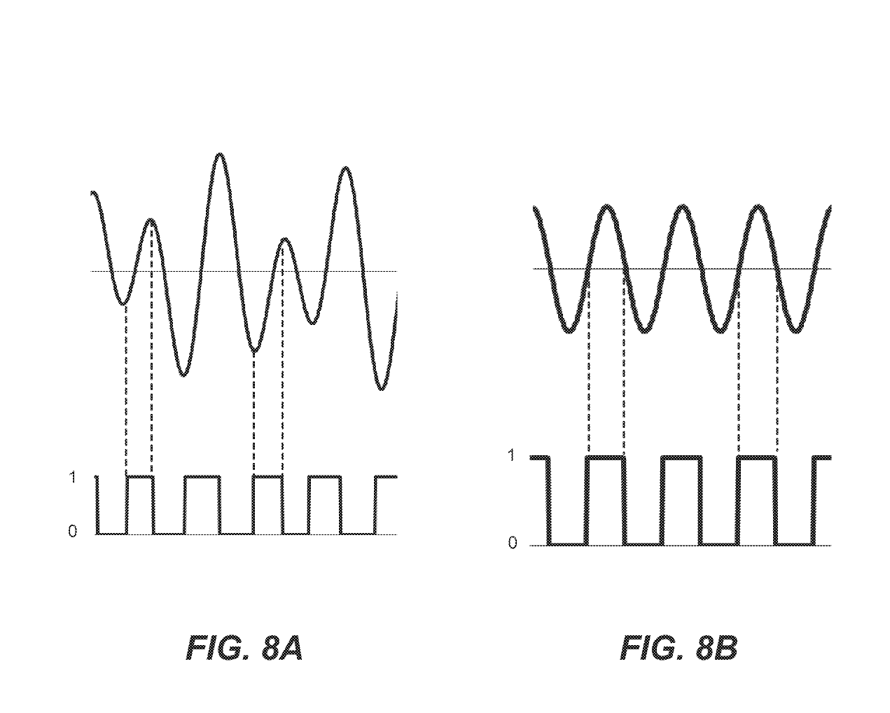

The binary conversion module 330 converts 520 the discrete derivative d[i] to a binary signal b[i] responsive to a comparison to a threshold (e.g., zero, a non-zero number). In the illustrated embodiment, the binary signal b[i] is one if the discrete derivative d[i] is positive and zero if the discrete derivative d[i] is zero or negative. Alternatively or additionally, higher order derivatives, or anti-derivatives, may be taken 510 in place of a discrete derivative. Data may be converted 520 to a binary signal without taking the discrete derivative. An example of converting recorded data to a binary signal is described with respect to FIG. 8.

The binary conversion module 330 windows 530 the binary signal b[i] into vectors of N samples. Samples from the binary signal b[i] from the index i=0 to the index i=N−1 are placed into a vector b_N[j] having N elements. Consequently, the vector b_N[j] contains the samples collected between the time associated with the sample at index 0 and the time associated with the sample at index N−1. The binary conversion module 330 repeats the windowing for every N samples in an embodiment. In the case of an approximate discrete Fourier transform, the windowing extracts the frequencies from the time period covered by the N samples in the window. Windowing 530 may be based on a period of time rather than a number of samples. The basis function multiplication module 335 then multiplies 430 the vector b_N[j] by one or more basis functions.

Referring to FIG. 6, illustrated is a process flow diagram for multiplying 430 binary data in the exemplary case of an approximate discrete Fourier transform. The basis function multiplication module 335 retrieves 610 basis functions from the basis function store 310. In an embodiment, the basis function multiplication module 335 retrieves 610A a matrix C[i,j] having N rows and N columns of binary cosine values for computing the real component of frequency amplitudes. A row i of the matrix C[i,j] corresponds to a binary cosine basis function at a frequency proportional to i. The entries of C[i,j] in column j give the value of the binary cosine basis functions at time j. Similarly, the basis function multiplication module 335 retrieves 610B a matrix S[i,j] having N rows and N columns of binary sine values. The basis function matrices may be generated rather than retrieved 610 from the basis function store 310. A graphical illustration of rows of an example basis function matrix is described with respect to FIG. 9.

The basis function multiplication module 335 performs 620 multiplications using an XNOR operation. The XNOR of A and B returns one when both A and B are one or when both A and B are zero. The XNOR of A and B returns zero when one of A and B is one and one of A and B is zero. In other words, the XNOR returns one when A and B are correlated and zero when A and B are uncorrelated, similar to a multiplication. The basis function multiplication module 335 performs the multiplication by taking the element-wise XNOR of the elements across a row of the basis function matrix C[i,j] or S[i,j] with the elements of the vector b_N[j]. The element-wise multiplication of the binary cosine matrix C[i,j] with the vector b_N[j] is repeated for all N rows of the binary cosine matrix C[i,j] to create a real product matrix P_real[i,j]. Similarly, the element-wise multiplication of the binary sine matrix S[i,j] with the vector b_N[j] is repeated for all N rows of the binary sine matrix S[i,j] to create an imaginary product matrix P_imag[i,j]. The product tallying module 340 then tallies 440 the entries across the rows of the real and imaginary product matrices.

In an alternative embodiment, the basis function multiplication module 335 performs 620 multiplications using an exclusive or (XOR) operation. The XOR of A and B returns one when one of A and B is one and one of A and B is zero. The XOR of A and B returns zero when both A and B are one or when both A and B are zero. In other words, the XNOR returns zero when A and B are correlated and one when A and B are uncorrelated.

Alternatively or additionally, the basis function multiplication module 335 retrieves 610 more or fewer basis function matrices from the basis function store 310 for multiplication. For example, an approximation of the discrete Hartley transform may use the cosine-and-sine (CAS) function (cos [i×j]+sin [i×j]) converted to binary as a basis function. The basis function multiplication module 335 retrieves 610 a matrix CAS[i,j] having rows corresponding to a binary cosine-and-sine function at increasing frequencies from the basis function store 310. The basis function multiplication module 335 performs 620 the XNOR between the retrieved matrix and the vector b_N[j], resulting in a product matrix.

Alternatively or additionally, the XNOR output values are shifted and/or scaled. For example, an alternative XNOR of A and B returns the value three when both A and B are one or when both A and B are zero; the alternative XNOR returns one when one of A and B are one. Alternatively or additionally, XNOR is generalized for non-binary input values. For example, values above a threshold are treated similar to an input of one in the conventional XNOR definition and values below a threshold are treated as an input of zero in the conventional XNOR definition. Alternatively or additionally, a combination of logical operations, such as NOT, AND, OR, NAND, and/or NOR is used to provide the functionality of an XOR or XNOR operation.

Referring to FIG. 7, illustrated is a process flow diagram for tallying 440 product matrices, in one embodiment. The product tallying module 340 receives a product matrix P[i,j] having N rows and N columns and splits the product matrix into two split product matrices. The first split product matrix P[i,0:N/2−1] contains the first half of columns in P[i,j] and the second split product matrix P[i,N/2:N−1] contains the second half of columns in P[i,j]. Because an XNOR is used to perform multiplication, the entries of the split product matrices are ones or zeros.

The product tallying module 340 performs 710 a bit sum on the split product matrices across each row of the split product matrix. In one embodiment, the bit sum algorithm adds one for each entry in a row containing a one and subtracts one for each entry in a row containing a zero. The resulting split sums s1[i] and s2[i] are column vectors having N entries, each entry corresponding to a bit sum of a row of a split product matrix. In one embodiment, a bit sum store 345 contains entries mapping product matrix rows to bit sum entries (e.g., a lookup table). The product tallying module 340 checks the bit sum store 345 repeatedly to obtain the bit sums of the rows of a split product matrix. An example lookup table used to perform a bit sum is described with respect to FIG. 10. In some embodiments, the bit sum across a row is performed without the use of a bit sum store 345 at increased computational cost relative to using a bit sum store 345.

The product tallying module 340 sums 720 the split sums s1[i] and s2[i] element by element into the bit sum result s[i], a column vector having N entries. The product tallying module 340 then sends 450 the bit sum result for storage or further processing. When multiple product matrices are received, multiple bit sum result vectors are computed. In some embodiments, the product matrix is not split, and the bit sum is performed across the rows of the product matrix, which obviates summing 720 the split sums. In some embodiments, the product matrix is split unevenly and/or is split into more than two matrices. The resulting sum corresponds to the strength of a frequency component of the initial recorded data, where the frequency component has the same frequency as the basis function.

When the data transform process 400 computes an approximation of the discrete Fourier transform, the product tallying module 340 tallies 440 the real product matrix and imaginary product matrix to create two bit sum results. The product tallying module 340 may tally 440 the real and imaginary product matrices in sequence or simultaneously (e.g., using duplicate hardware). The bit sum results include a real bit sum result from the real product matrix and an imaginary bit sum result from the imaginary product matrix. Entry i of the bit sum result corresponds to the amplitude of the frequency component represented in row i of the basis function matrix. For example, when computing an approximation of the discrete Fourier transform, row i of the binary cosine matrix has a particular frequency. The approximate real component of the amplitude of that particular frequency in the raw data signal is given by entry i of the real bit sum result; the approximate imaginary component of the amplitude of that particular frequency in the raw data signal is given by entry i in the imaginary bit sum result.

Referring to FIGS. 8A and 8B, example processes are illustrated for converting data to a binary signal in accordance with one embodiment. FIG. 8A illustrates converting 420 example raw data 810 to binary raw data 820, as is described in FIG. 5. The binary conversion module 330 takes 510 the derivative of data and compares the derivative to zero. In other words, the example embodiment of the binary conversion module 330 converts 420 data having a positive derivative to one and data having a non-positive derivative to zero. As illustrated in FIG. 8A, the binary raw data 820 is one when the example raw data 810 is increasing (i.e., the derivative is positive) and zero otherwise.

FIG. 8B illustrates converting a cosine 830 to a binary cosine 840 based on a threshold. In an embodiment, the basis function module 325 converts 420 basis functions to a binary representation according to a threshold. The binary cosine 840 is one when the cosine 830 is positive; otherwise the binary cosine 840 is zero.

Referring to FIG. 9, illustrated are example basis functions from the basis function store 310. In an embodiment, the basis function store 310 contains basis functions 910, 920, 930, and 940 based on a cosine converted to a binary representation as described previously. The basis function 910 is a single period of a binary cosine. The basis function 920 is a binary cosine having twice the frequency of the basis function 910. Similarly, the basis functions 930 and 940 are binary cosines having three and four times the frequency of the basis function 910, respectively. In an embodiment, the basis function store 310 contains additional basis functions. In an embodiment, the basis function store 310 has N basis functions, where N is equal to the number of samples selected when the data is windowed 530. The basis functions are stored in a matrix having N rows and N columns. Each row stores a basis function. The columns correspond to N evenly spaced intervals of time. An entry of the matrix in a particular row and column contains the value of the basis function corresponding to the particular row at the time corresponding to the particular column. In one embodiment, the basis function multiplication module 335 uses the example binary cosine basis functions to compute the real part of a binary Fourier transform. In an embodiment, the basis function store 310 may be implemented as a lookup table as hardware or firmware. Alternatively or additionally, the lookup table is stored in random-access memory (RAM).

Referring to FIG. 10, illustrated is an example embodiment of the bit sum store 345. In one embodiment, the bit sum store 345 is implemented as a lookup table. In response to receiving the input bits 1005 from the product tallying module 340, the bit sum store 345 returns a corresponding bit sum 1010. For example, the example entry 1015 returns a bit sum 1010 of negative one when the received input contains the input bits 1005 zero, one, and zero. The bit sum is performed using a different weighting of zero and one entries. For example, an alternative bit sum adds two for every entry containing a one in a row and subtracts zero for every entry containing zero in a row. The illustrated embodiment may perform a discrete Fourier transform with a window length of three samples, but other embodiments may have additional or fewer entries based on the number of entries in the input to the bit sum store 345.

In an embodiment where the basis function multiplication module 335 uses an XNOR operation to multiply the binary data against a binary basis function, an output of one from the basis function multiplication module 335 (and therefore an input bit 1005 of one) corresponds to a correlation between the binary data and the binary basis function. As the number of correlations between the binary data and the binary basis function increases, the value of the bit sum 1010 increases (when determining the bit sum 1010 with the lookup table illustrated in FIG. 10). Accordingly, a bit sum 1010 with a highly positive value corresponds to a strong overall degree of correlation between the binary data and the binary basis function. In other words, a highly positive bit sum 1010 indicates that the basis function used to derive the binary basis function is a dominant component of the input data converted to the binary data.

In an embodiment where the basis function multiplication module 335 uses an XOR operation to multiply the binary data against a binary basis function, an output of zero from the basis function multiplication module 335 (and therefore an input bit 1005 of zero) corresponds to a correlation between the binary data and the binary basis function. As the number of correlations between the binary data and the binary basis function increases, the value of the bit sum 1010 decreases (when determining the bit sum 1010 with the lookup table illustrated in FIG. 10). Accordingly, a bit sum 1010 with a highly negative value corresponds to a strong overall degree of correlation between the binary data and the binary basis function. In other words, a highly negative bit sum 1010 indicates that the basis function used to derive the binary basis function is a dominant component of the input data converted to the binary data.

The description of data in a “vector,” a “matrix,” or an “array” having “entries” in “rows” or “columns” is for purposes of illustration. Alternative embodiments may organize data in various data structures and/or may process data in different sequences or orders. Description of data as “binary” or “integral” is for purposes of illustration. Alternatively or additionally, data may be represented in various digital or analog formats (e.g., as a floating point number).

Alternatively or additionally, the disclosed embodiment of a binarized data transform may be generalized to data having a higher dimensionality. For example, to perform an approximation of a two-dimensional Fourier transform, the binary conversion module 330 converts 420 two-dimensional input data to a binary signal by comparing a generalized derivative (e.g., the magnitude and/or direction of a two-dimensional discrete gradient) of the input data to a threshold. The basis function store 310 contains two-dimensional binary sine functions and cosine functions for multiplication using the XNOR or XOR operation. The product tallying module 340 recursively sums the resulting products to create one two-dimensional output of real amplitudes and another of imaginary amplitudes. Such an embodiment may be used as part of frequency component extraction from an image, for example.

### Additional Considerations

The disclosed embodiments beneficially allow for efficient processing of a data transform. The use of binary signals decreases the memory necessary and consequently reduces the hardware necessary for implementation, thereby providing advantages such as reduced production cost and/or manufacturing complexity. The use of a XOR or XNOR operation increases processing speed and/or required implementation hardware compared to using a normal multiplication. Using the basis function store 310 obviates the need to repeatedly perform calculations to generate basis functions, which may require multiplications, divisions, or exponentiation, or trigonometric or other transcendental operations. These operations could require additional hardware, so the basis function store 310 reduces necessary hardware for implementation. Using the bit sum store 345 obviates the need for repeated additions. Splitting the bit sum operation reduces the necessary entries in the bit sum store 345, which can reduce hardware necessary for implementation.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of components, modules, or mechanisms, for example, as illustrated in FIGS. 1, 2, and 3. Modules may constitute either software modules (e.g., code embodied on a machine-readable medium or in a transmission signal) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

The various operations of example methods described herein may be performed, at least partially, by one or more processors, e.g., processor 125, that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs).)

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for transforming sensor data through the disclosed principles herein. Thus, while particular embodiments and applications have been illustrated and described, it is to be understood that the disclosed embodiments are not limited to the precise construction and components disclosed herein. For example, references to particular modules are for purposes of illustration. Alternative embodiments may use additional or fewer modules or alternative configurations of modules. Various modifications, changes and variations, which will be apparent to those skilled in the art, may be made in the arrangement, operation and details of the method and apparatus disclosed herein without departing from the spirit and scope defined in the appended claims.

1-20. (canceled)

21. A method for extracting frequency information from data sampled over a period of time, the method comprising:
collecting data comprising a set of samples;
converting the collected set of samples to a set of binary values by comparing derivatives of the set of samples to a threshold conversion value, each binary value corresponding to a particular time of the period of time;
obtaining a set of basis function values of a basis function having a frequency, each basis function value corresponding to the particular time of each binary value;
multiplying, by one or more processors, the set of binary values with the set of basis function values into a set of products;
determining a result representing approximate strength of the frequency of the basis function in the set of samples by tallying the set of products; and
providing the result to a component of a portable device.

22. The method of claim 21, wherein multiplying the set of binary values with the set of basis function values comprises:
determining the set of products using at least one of: (a) an exclusive nor (XNOR) operation between each binary value and the basis function value corresponding to the particular time of each binary value, and (b) an exclusive or (XOR) operation between each binary value and the basis function value corresponding to the particular time of each binary value.

23. The method of claim 21, wherein determining the result by tallying the set of products comprises:
separating products into a plurality of groups, each group containing one or more products;
applying a bit sum function to the one or more products in each group to compute a plurality of intermediate totals; and
summing the plurality of intermediate totals to determine the result.

24. The method of claim 23, wherein applying the bit sum function comprises:
instantiating an intermediate total of the plurality of intermediate totals, the intermediate total having a starting value;
adding a first weight to the intermediate total for each product exceeding a bit sum threshold; and
subtracting a second weight from the intermediate total for each product not exceeding the bit sum threshold.

25. The method of claim 21, wherein determining the result by tallying the set of products comprises:
consulting a lookup table, the lookup table storing bit sums corresponding to different sets of products.

26. The method of claim 21, repeated for a plurality of frequencies of the basis function to determine a plurality of results representing strengths of a plurality of frequencies of the basis function in the set of samples.

27. The method of claim 21, wherein the basis function is based on at least one of: a sine function, a cosine function, a wavelet, a binary form of a sine function, a binary form of a cosine function, a binary form of a wavelet, and a linear combination thereof.

28. A computer program product comprising a tangible non-transitory computer-readable medium storing computer program instructions for extracting frequency information from data sampled over time, the computer program instructions executable by a processor to:
convert the collected set of samples to a set of binary values by comparing derivatives of the set of samples to a threshold conversion value, each binary value corresponding to a particular time of the period of time;
obtain a set of basis function values of a basis function having a frequency, each basis function value corresponding to the particular time of each binary value;
multiply the set of binary values with the set of basis function values into a set of products;
determine a result representing approximate strength of the frequency of the basis function in the set of samples by tallying the set of products; and
provide the result to a component of a portable device.

29. The computer-readable medium of claim 28, wherein the computer program instructions executable by the processor to multiply the set of binary values with the set of basis function values comprise computer program instructions executable by a processor to:
determine the set of products using at least one of: (a) an exclusive nor (XNOR) operation between each binary value and the basis function value corresponding to the particular time of each binary value, and (b) an exclusive or (XOR) operation between each binary value and the basis function value corresponding to the particular time of each binary value.

30. The computer-readable medium of claim 28, wherein the computer program instructions executable by the processor to determine the result by tallying the set of products comprise computer program instructions executable by the processor to:
separate products into a plurality of groups, each group containing one or more products;
apply a bit sum function to the one or more products in each group to compute a plurality of intermediate totals; and
sum the plurality of intermediate totals to determine the result.

31. The computer-readable medium of claim 30, wherein the computer program instructions executable by the processor to apply the bit sum function comprise computer program instructions executable by the processor to:
instantiate an intermediate total of the plurality of intermediate totals, the intermediate total having a starting value;
add a first weight to the intermediate total for each product exceeding a bit sum threshold; and
subtract a second weight from the intermediate total for each product not exceeding the bit sum threshold.

32. The computer-readable medium of claim 28, wherein the computer program instructions executable by the processor to determine the result by tallying the set of products comprise computer program instructions executable by the processor to:
consult a lookup table, the lookup table storing bit sums corresponding to different sets of products.

33. The computer-readable medium of claim 28, further comprising computer program instructions executable by the processor to repeat execution of the computer program instructions for a plurality of frequencies of the basis function to determine a plurality of results representing strengths of a plurality of frequencies of the basis function in the set of samples.

34. The computer-readable medium of claim 28, wherein the basis function is based on at least one of: a sine function, a cosine function, a wavelet, a binary form of a sine function, a binary form of a cosine function, a binary form of a wavelet, and a linear combination thereof.

35. A system for extracting frequency information from data sampled over time, the system comprising:
a sensor collecting samples of data, each sample taken at a particular time;
one or more processors configured to execute instructions and to receive data from the sensor; and
a non-transitory computer-readable medium storing computer program instructions executable by the one or more processors, the computer program instructions comprising instructions to:
convert the collected set of samples to a set of binary values by comparing derivatives of the set of samples to a threshold conversion value, each binary value corresponding to a particular time of the period of time;
obtain a set of basis function values of a basis function having a frequency, each basis function value corresponding to the particular time of each binary value;
multiply the set of binary values with the set of basis function values into a set of products;
determine a result representing approximate strength of the frequency of the basis function in the set of samples by tallying the set of products; and
provide the result to a component of the system.

36. The system of claim 35, wherein the computer program instructions to multiply the set of binary values with the set of basis function values comprise computer program instructions to:
determine the set of products using at least one of: (a) an exclusive nor (XNOR) operation between each binary value and the basis function value corresponding to the particular time of each binary value, and (b) an exclusive or (XOR) operation between each binary value and the basis function value corresponding to the particular time of each binary value.

37. The system of claim 35, wherein the computer program instructions to determine the result by tallying the set of products comprise computer program instructions to:
separate products into a plurality of groups, each group containing one or more products;
apply a bit sum function to the one or more products in each group to compute a plurality of intermediate totals; and
sum the plurality of intermediate totals to determine the result.

38. The system of claim 37, wherein the computer program instructions to apply the bit sum function comprise computer program instructions to:
instantiate an intermediate total of the plurality of intermediate totals, the intermediate total having a starting value;
add a first weight to the intermediate total for each product exceeding a bit sum threshold; and
subtract a second weight from the intermediate total for each product not exceeding the bit sum threshold.

39. The system of claim 35, wherein the computer program instructions to determine the result by tallying the set of products comprise computer program instructions to:
consult a lookup table, the lookup table storing bit sums corresponding to different sets of products.

40. The system of claim 35, further comprising computer program instructions to repeat the computer program instructions for a plurality of frequencies of the basis function to determine a plurality of results representing strengths of a plurality of frequencies of the basis function be used to approximate various transforms.
